(19) United States
(12) Patent Application Publication
(10) Pub. No.: US 2004/0077017 A1
(43) Pub. Date: Apr. 22, 2004

(54) Detection methods

(76) Inventors: Amelie Karlstrom, Stockholm (SE); Per-Ake Nygren, Ekero (SE)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/250,927

(22) PCT Filed: Jan. 14, 2002
(86) PCT No.: PCT/GB02/00127

(30) Foreign Application Priority Data
Jan. 12, 2001 (GB) 0100919.0
May 8, 2001 (CA) 2,346,583

(51) Int. Cl. G01N 33/53; G01N 33/543

(52) U.S. Cl. 435/7.1; 436/518

(57) ABSTRACT

The present invention relates to methods of detecting the presence of target molecules in a sample, in particular protein target molecules. In particular, the invention provides a method of detecting the presence of a target molecule in a sample which comprises contacting said sample with a detector protein, said detector protein being a binding partner for said target molecule and being derivatized by two fluorescent reporter groups, the energy transfer between said reporter groups undergoing a detectable change on binding of the target molecule to said detector protein and a detector protein molecule having a binding site for a target molecule and having covalently attached thereto two fluorescent reporter groups, the energy transfer between said reporter groups undergoing a detectable change on binding of the target molecule to said detector protein. The detector protein comprises or consists f a combinatorial protein.

The present invention relates to methods of detecting the presence of target molecules in a sample, in particular protein target molecules. More particularly, the invention relates to such detection methods which rely on association between the target molecule and a binding partner.

In recent years, great advances in DNA microarray technologies have facilitated high throughput parallel analysis of gene expression. This type of experiment has yielded large amounts of valuable data, but shortcomings of the method have nevertheless been demonstrated. It is clear that important changes in cellular states, e.g. from healthy to diseased, are not necessarily best characterized by changes in mRNA levels. The protein abundance of a cell has been shown to correlate poorly with the corresponding mRNA levels, and the activity of proteins is frequently regulated by post-translational modifications, rather than just by alteration of the expression level. In many cases it would be preferred, if reproducible and sensitive methods were available, to analyze biological samples at the level of protein.

Specific detection of proteins is an important aspect of proteomic studies, the presently used techniques are each associated with problems that limit the possible applications. Mass spectrometric techniques have been developed for the analysis and identification of proteins eluted from two-dimensional gels or bound to chips and these methods have been shown to be highly sensitive and able to detect very small quantities of protein. However, mass spectrometry is not a quantitative technique and unless the proteins are labelled with probes that enable quantitative analysis, the relative amounts of different proteins present in a sample cannot be determined. Fluorescent methods that rely on labelling the protein population in a sample before analysis for a direct readout, suffers from the drawback that dissimilar proteins are labelled to different degrees, which results in a highly variable signal for different proteins and limits its use in quantitative measurements. In addition, labelling and preparation of the protein sample is a time-consuming step that preferably is avoided.

Preparation of protein arrays analogous to the DNA microarrays used for analysis of gene expression is accompanied by several technical difficulties. In contrast to mRNA, which can be amplified by RT-PCR and analysed indirectly at the DNA level, proteins cannot be amplified by any known method and a highly sensitive method for detection is therefore required. Fluorescent or radiometric techniques typically have high sensitivity, but whereas oligonucleotides are easily, labelled by coupling of fluorescent or radioactive nucleotides, uniform and reproducible labelling of a protein population is less straightforward. Another problem is the capture of the proteins to be analysed, since specific, high-affinity binders are only available for a fraction of the protein present in a cell.

Due to the high sensitivity of fluorescent techniques and the wide range of fluorescent probes available, fluorescent methods are widely used in biochemistry and cell biology. A number of methods have been developed that are based on the concept of fluorescence resonance energy transfer (FRET), which is a non-radiative induced dipole-induced dipole mechanism for transfer of energy from an excited donor fluorophore to a proximal acceptor molecule (Förster, 1948). Reference herein to “energy transfer” is a reference to FRET. The theory of FRET was first described by Förster, who showed that the efficiency of the energy transfer, E, is highly dependent on the distance, R, between the two groups. Since FRET occurs over distances 10-100 Å, it is particularly useful for measuring intramolecular distances and processes on a cellular scale.

Conformation-dependent biosensors based on the principle of FRET have recently been developed, e.g. to analyze intracellular Ca²⁺ concentration (Miyawaki et al, 1997), measure phosphorylation of the Crk-II adapter protein (Cotton & Muir, 2000), and detect interactions between the GTP-binding protein Cdc42 and its effector proteins (Nomanbhoy & Cerione, 1999). Homogenous FRET-based immunoassays for detection of the specific interaction between an antibody and an antigen have also been described (Ueda et al., 1999 and Arai et al, 2000). In the assays described by Ueda and Arai et al., the complex formed between different fluorescently-labelled V_{H} and V_{L} fragments is stabilized upon binding of antigen, and the binding is monitored by an increase in the efficiency of energy transfer between the labelled antibody half-fragments. Labelling of the proteins was performed either by coupling of fluorophores via amine groups or by gene fusion to two different fluorescent reporter proteins.

In many of these previously described systems, complicated fusion proteins comprising a number of different domains including fluorescent reporter domains are generated. For example, Miyawaki et al. describe the use of pairs of mutants of green fluorescent protein (GFP) fused to a Ca²⁺ binding calmodulin domain. It would be desirable if a simpler and smaller molecule could be generated, not least because the use of fused reporter proteins may interfere with binding to a target molecule. Cotton et al describe a system where small fluorophores are used, fluorescein and tetramethylrhodamine, but again a complex fusion protein of different subunits is constructed. Certain prior art systems typically involve 2 molecules which are individually labelled and are only able to undergo FRET which they both associate with a third molecule.

An alternative strategy would be to utilize fluorescent detection in a reversed format, where the formation of a binding protein-target complex leads to a decrease in the efficiency of FRET.

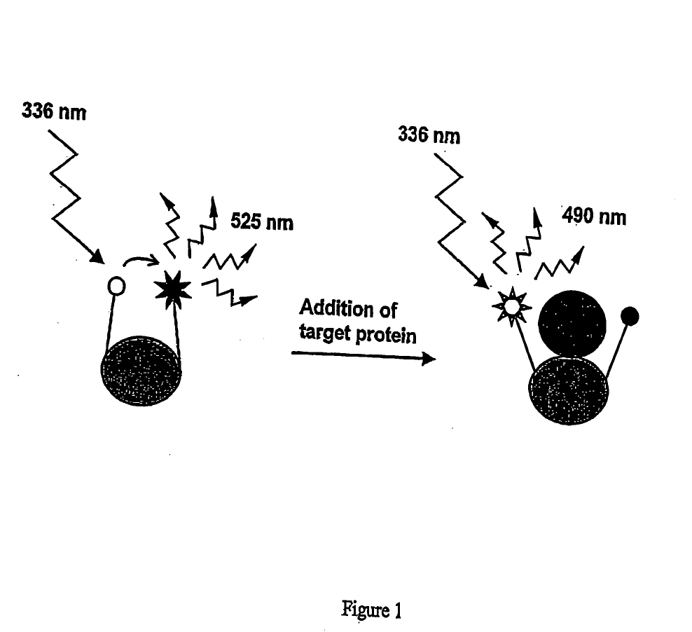

According to this invention, a novel approach for detection of unlabelled proteins is presented, facilitating the production of protein microarrays. Detection of protein-protein interactions mediated by a protein A-derived binding protein covalently labelled with a fluorescent donor/acceptor pair suitable for fluorescence resonance energy transfer has been investigated. When the donor and the acceptor groups are in close proximity to each other, energy from the excited donor group can be transferred to the acceptor group, but as the protein binds to its target protein, the fluorescent signal is altered (see FIG. 1). Since only the binding protein is derivatized with the fluorophores, labelling of the sample to be analysed is circumvented.

Thus, according to one aspect, the present invention provides a method of detecting the presence of a target molecule in a sample which comprises contacting said sample with a detector protein, said detector protein being a binding partner for said target molecule and being derivatized by two fluorescent reporter groups, the energy transfer between said reporter groups undergoing a detectable change on binding of the target molecule to said detector protein.

Detection typically involves measuring the emission from one or both of the fluorescent reporter groups. Thus, through detecting changes in emission, so the presence of a target molecule is indicated.

The reporter groups are preferably non-proteinaceous and thus do not need to be incorporated as reporter domains into the detector protein (as would be the case, e.g. with GFP derivatives) but are small molecules which can be added as labels to generate a detector protein. Suitable reporter groups are fluorophores, such am derivatives of fluorescein, rhodamine, eosin, erythrosin, coumarin, naphtalene, pyrene, pyridyloxazole, benzoxadiazole and sulfoindocyanine. For efficient conjugation to the protein, the fluorophores are preferably in the form of amine- or thiolreactive reagents, such as isothiocyanates, succinimidyl eaters, aldehydes, sulfonyl halides, alkyl halides, haloacetamides, maleimides, azirdines or epoxides. Suitable examples include rhodamine B sulfonyl chloride and fluorescein maleimide, N-iodoacetyl-N′-(5-sulfo-1naphtyl) ethyl-enediamine (1,5-IAEDANS) or iodoacetamide and succinimidyl 6-(N-((7-nitrobenz-2-oxa-1,3-diazol-4-yl)amino)hexanoate (NBD-X, SE), (diethylamino)coumarin (DEAC) or an N-methyl-anthraniloyl deoxyguanine nucleotide (e.g. MantdGDP or MantdGTP) and sNBD (succinimidyl 6-[(7-nitrobenz-2-oxa-1,3-diazol-4-yl)amino]hexanoate, also known as “NBD-X,SE”).

Any target molecule which is capable of inducing a conformational change in a binding protein may be analysed according to the method described herein. Preferably, the target molecule is a protein molecule or derivative or fragment thereof, a polypeptide or peptide. Typically, the target molecule will be between 10 and 1000 amino acids in length, e.g. 20 to 500.

Libraries of binding proteins can be generated and screened for their suitability for binding a given target molecule. Once selected, the binding protein can be used to generate a detector protein by labelling with fluorescent reporter groups, optionally with the additional step of modifying the primary structure of th binding protein to incorporate an amino acid to which a reporter group may be attached.

Thus, in a further aspect, the present invention provides a method of preparing a detector protein for use in the detection of a target molecule in a sample, said method comprising;

(a) selection, form a library of molecules, of a binding protein capable of binding to said target molecule,

(b) optionally introducing into the protein identified in step (a) one or more amino acid residues which can be derivatized by a fluorescent reporter group (this will preferably be achieved by site directed mutagenesis of the nucleic acid encoding the protein identified in step (a)), and

(c) labelling the binding protein either simultaneously or preferably sequentially with two fluorescent reporter groups.

The introduced amino acid is preferably internal, i.e. is not at the N or C terminus and it will typically be unique, the only amino acid in the binding protein of that type.

The detector protein preferably is or comprises a single domain binding protein which is capable of binding to the target molecule and undergoing a conformational change which causes a measurable increase or decrease in the energy transfer between two fluorescent reporter groups which are carried by the single domain binding protein. This simple single domain binding protein is therefore derivatized at two positions to report on target binding and itself responsible for binding to the target. This is a much simpler arrangement than the multi-domain (fusion protein) structures of the prior art where a domain may be responsible for reporting or binding but not both functions.

In alternative embodiments of the invention, the detector protein may comprise two or more, preferably 2 different domains which bind to non-overlapping epitopes on a target protein. Thus each domain is still performing two roles, that of target recognition and binding, and as each domain carries a fluorescent reporter group, that of reporting. A flexible linker could be used to separate the domains so that FRET does not occur (or is at a low level), unless the two domains are both bound to their respective epitopes on the target molecule.

In a variation of such a system which provides a further aspect of the invention, pairs of anti-idiotypic protein binding domains (affibodies) have been generated which bind to each other through their variable regions. Typically, one of the pair is a target specific affibody to which a second affibody has been raised which is capable of binding to the binding surface of the first affibody. In a competitive type system, the target molecule separates the two domains from each other, typically causing a significant decrease in FRET. Each anti-idiotypic affibody is thus labelled with a fluorescent reporter group and they may be linked in a single protein or used as free domains. In a further aspect, two identical or at least functionally equivalent labelled domains could be used if the target is a homodimer and, significant FRET would only occur when the two domains are bound to the target.

A domain is a readily identifiable unit of a protein molecule whose secondary and/or tertiary structure distinguishes it from other parts of the molecule, i.e. it is structurally independent.

The double derivatized binding domain (which may or may not constitute the entire detector protein) is typically no more than about 200 amino acids in length e.g. 50-150 amino acids in length. This binding domain is preferably at least 25 amino acids in length. Any binding domain for use according to the present invention is preferably folded which has the benefits of a higher potential affinity for a target molecule (thermodynamic effects, less entropy lose on binding), proteolytic stability and structural integrity/resilience which means that it in more likely than an unfolded domain to remain active when attached to a solid phase or fused to other domains.

Preferably the detector protein comprises or consists of a combinatorial protein, which can be defined as a protein which is not naturally occurring but generated through the introduction of random alterations into the primary sequence of a target protein. These alterations typically being introduced at the nucleic acid level. Such combinatorial proteins and libraries of different combinatorial proteins can be readily screened for binding or other functionalities. Combinatorial protein engineering techniques and methods of screening these proteins are well known in the art. Preferred combinatorial detector proteins are those which are derived from staphylococcal protein A, in particular the B domain thereof. Where the detector protein is made up of more than one domain, one or more, e.g. all of the domains may be combinatorial protein domains.

By “binding partner” is meant that the detector protein is able to bind in a specific manner to the target molecule. In other words, there is binding over and above general electrostatic or similar associations. The relationship between these two moieties need not be exclusive, in that the target molecule may be capable of binding to more than one type of detector protein. The detector protein may be capable of binding to more than one target molecule but preferably it will have a much greater affinity (e.g. at least 6 fold, preferably at least 10 fold e.g. greater affinity) for the target molecule than any other molecule in the sample to be tested. The labelled detector protein can be considered to be ‘actively’ involved in recognition of the target molecule rather than a passive partner which is itself recognised by the target.

The “sample” to be tested may be any sample which could contain a target molecule of interest. The sample may be biological, e.g. taken from a plant or human or animal body, typically derived from a body fluid, such as blood or urine or an environmental sample e.g. water, soil or food. In addition, the sample may also be taken from a cell or tissue material or culture medium including, but not limited to, whole cell fractions, intracellular fractions, nuclear fractions, periplasmic, fractions, membrane fractions and organelle fractions.

The energy transfer (FRET) between the reporter groups may increase and this would result in greater emission by the acceptor group or it may decrease which would typically result in greater emission by the donor group.

Thus a binding protein has been derivatized at defined molecular positions with donor and acceptor reporter groups, that are affected by the presence of a completed target substance. This has been achieved through controlled and site-specific double-labelling of a single domain binding protein. Preferably, at least one of the reporter groups is not situated at the N or C terminus (either to the actual N or C terminal residues or the residues adjacent thereto) of the detector protein but is attached to an internal amino acid. Thus an ‘internal’ residue is not immediately adjacent to the N or C terminal residues and is preferably not one of the 3 residues at either end, more preferably not one of the 5 residues at either end of the binding domain. We prefer the use of at least one internal residue because there is no intention for the two reporter moieties to dimerise, instead it is preferred for the reporter moieties to ‘communicate’ through FRET without more direct physical association. As discussed below, the internal amino acid to which the report group is attached is preferably unique in the molecule (and may have been introduced into the native molecule e.g. by site-directed mutagenesis) so the exact position of the reporter group can be guaranteed as there is only one suitable site for attachment.

Binding proteins based on scaffolds of more simple structure than antibodies are attractive candidates for site-specific labelling due to the potential of introducing unique sites, such as cysteine residues, suitable for selective coupling chemistry. One such class of binding proteins are denoted affibodies and are based on the single domain immunoglobulin (Fc)-binding staphylococcal protein A analogue Z, which lacks cysteine residues and is a robust, 58-residue three-helix bundle structure. This parental domain has been exploited as a scaffold for combinatorial protein engineering efforts to obtain novel affinity proteins capable of selective recognition of a variety of target proteins other than the native Fc binding partner (Nord et al, 1995, Nord et al, 1997, Gunneriusson et al, 1999, Hansson et al, 1999). The proven high stability of the proteins based on the Z domain scaffold (Nord et al. 2000), would likely be an advantage in the preparation of protein arrays, where denaturation at the protein-surface interface and protein degradation are potential problems.

Any protein with a capability of recognising a target should be useful for derivatisation and use according to the invention. This includes antibodies and fragments thereof, originating from natural sources or produced by recombinant means. Examples of other domains suitable for engineering binding specificities and subsequent fluorophore labelling can for example be found among domains within bacterial receptor structures called receptins (Kronvall et al.)

In order to illustrate the principle of the present invention, a method is now described where a binding protein is chemically derivatized with two fluorescent reporter groups, which upon binding of the target protein show a dose-dependent shift in emission ratio that can be used to quantitatively determine the presence of the specific target protein. The fluorescent groups constitute a donor/acceptor pair, which in the doubly labelled protein is expected to undergo fluorescence resonance energy transfer when excited at the excitation maximum of the donor fluorophore. Transfer of energy between a donor and an acceptor group can be experimentally detected by a decrease in the fluorescence emission of the donor, a decrease in the lifetime of the excited state of the donor, or an increase in the fluorescence emission of the acceptor. Methods for the detection of fluorescence are well known in the art and examples may be found in the references cited herein. In this study, enzymatic. digestion of the donor/acceptor-labelled protein dramatically shifts the fluorescence emission spectra in favour of donor emission, which strongly suggests that fluorescence resonance energy transfer does occur in the intact, labelled protein, leading to a decrease in donor emission and an increase in acceptor emission.

Addition of target protein to the donor/acceptor-labelled binding protein leads to an increase in donor fluorescence and a decrease in acceptor fluorescence. This could be due to impaired transfer of energy from the excited donor fluorophore to the acceptor fluorophore in the protein-protein complex. It is possible the distance between the two fluorescent reporter groups is increased in the presence of target protein, either because the bulk of the target protein sterical interferes with the motion of the fluorophores, which are likely flexible in the unbound state, or because of a conformational change of the protein in the bound state. It is known that changes in distance between two groups of a donor/acceptor pair strongly affects the efficiency of the energy transfer. The sensitivity is greatest at distances close to the Förster distance R₀, which is a characteristic of the fluorophore pair and depends on the spectral overlap of the donor emission and acceptor absorption, the quantum efficiency of the donor, the refractive index of the medium and the relative orientation of the two interacting dipoles. Since the estimated distance across the binding surface of the B domain is about 30 Å and R₀ values typically are in the range of 20-60 Å, even small changes in distance are likely to affect the efficiency of energy transfer in this case. The shift in fluorescence emission ratio could also be an effect of the change in the local environment of the fluorophores, which could result in a modification of the fluorophore quantum yields.

The different effects described herein on the fluorescence emission ratio by the addition of the target protein Fc₃₍₁₎ and the non-binding, control protein Fc₃, show that regardless of the mechanism, the shift in emission ratio only occurs in the presence of a protein that specifically binds to the labelled protein. Since the fluorescence emission ratio increases with increasing concentration of target protein, the assay can be used to quantify the amount of protein in a sample. In this homogenous assay the sensitivity for detection of Fc₃₍₁₎ is in the same concentration range as the dissociation constant determined for the interaction between the Z domain and the Fc region of IgG (K_{D}~70 nM) It is possible that concentrating the sample in a small volume on the surface of a chip, where the protein analyte is captured from the surrounding medium, can further increase the sensitivity of the assay (silzel et al, 1998). Formation of the protein-protein complex and development of the fluorescent signal is sufficiently fast that the method should be possible to apply to high-throughput analyses of proteins. Since the method is label-free and circumvents preparation of the sample to be analyzed, it would be particularly suitable for parallel analysis of a large number of proteins.

As a model system , the interaction between the B domain of protein A and the Fc region of IgG was studied. However, the invention relates to a general concept that could be used to detect the interaction between other binding proteins and their respective target proteins. Specific binders against a wide range of proteins have been selected from a combinatorial protein library based on the scaffold of the synthetic Z domain, which is a synthetic analogue of the B domain (Nilsson et al, 1987). These binders, so-called affibodies, are small, compact proteins that lack naturally occurring cysteine residues and are easily expressed in bacteria. It has been shown by CD spectroscopy that the affibodies retain the overall three-dimensional fold of the Z domain (Nord et al, 1995) and these affibodies are thus suitable for use in the strategies developed here.

In the Examples herein, ABD (albumin binding domain) was used as an affinity tag to facilitate purification of the protein, it does not contain any cysteine residues and therefore is not itself labelled. Such a tag need not be included in the construct or it could be cleaved off before labelling.

Given the diversity of the target proteins for which affibodies have successfully been selected, it will be possible to select binders against other target proteins as well. The combination of an efficient method for generating high affinity binders and a method for label-free detection of the interaction between the binding protein and its target protein, forms the basis for a new approach that will be explored for the preparation of microarrays for global analysis of proteins.

According to a further aspect of the present is provided a detector protein molecule having a binding site for a target molecule and having covalently attached thereto two fluorescent reporter groups, the energy transfer between said reporter groups undergoing a detectable change on binding of the target molecule to said detector protein. Preferred features of this detector protein are discussed above in the context of the detection methods of the invention.

According to a further, related aspect of the invention, a shift in the fluorescence emission from a single fluorophore could be monitored as a consequence of a change in its local environment induced by binding of the target molecule to the detector protein. Thus a binding domain derivatised by a single fluorescent reporter group can act as a detector protein due to the modification in quantum yield from the fluorophore which can be monitored.

Reference is made herein to a ‘single domain’ binding protein. This protein may be ‘single domain’ in that the part responsible for detection and which has the reporter groups it a single domain but the whole protein actually used in the assay may be a fusion protein with another domain or domains not involved directly in detection.

The invention will now be described by the following Examples in which reference is made to the attached figures.

FIG. 1 Schematic picture of the interaction between the fluorescence-labelled binding protein and its target protein.

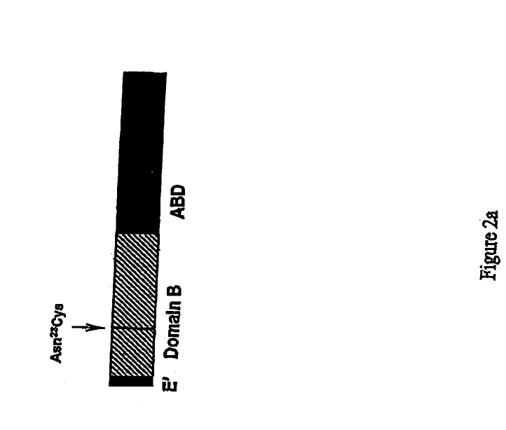

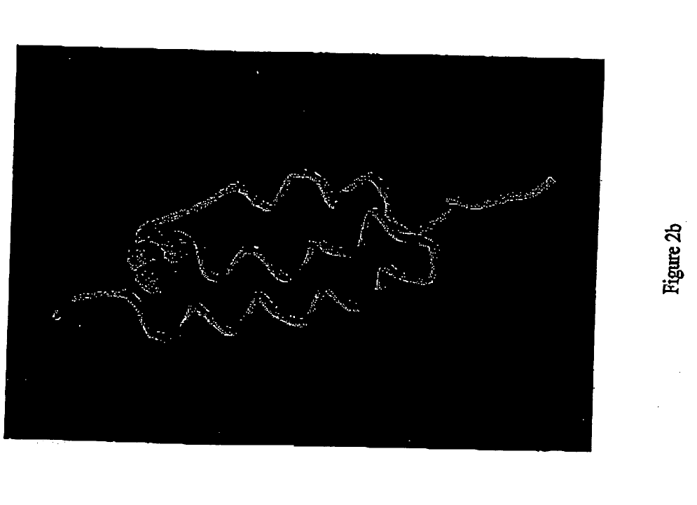

FIG. 2a) Fusion protein encoded by the expression vector. b) Model of the B (N²³C) mutant based on the three-dimensional structure of the B domain (Gouda et al, 1992) using the SYBYL 6.6 software (Tripos, Inc., St Louis, Mo.).

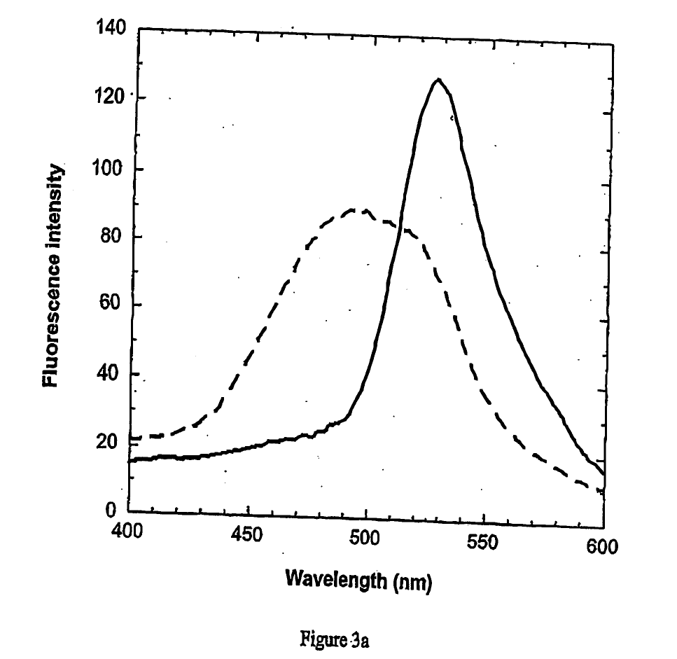

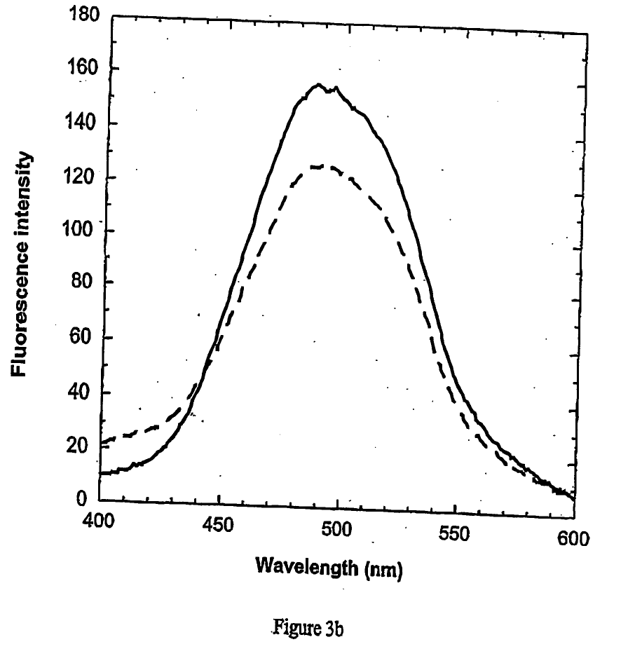

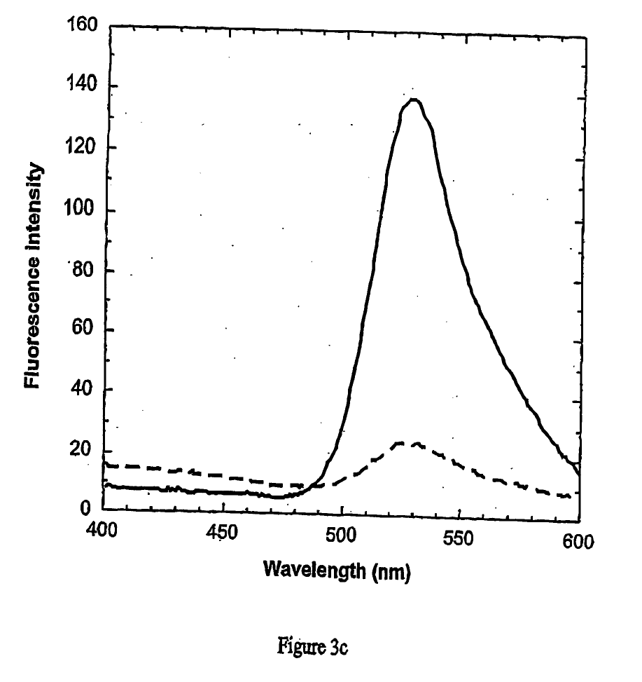

FIG. 3a) Emission spectra of EDANS/NBDX-labelled B(N²³C)-ABD in PBS, pH 7.4, before (----) and after ( - - - - ) digestion with Proteinase K.

b) Emission spectra of EDANS-labelled B (N²³C)-ABD in PBS, pH 7.4, before (----) and after ( - - - - ) digestion with Proteinase K.

c) Emission spectra of NBDX-labelled B(N²³C)-ABD in PBS, pH 7.4, before (----) and after ( - - - - ) digestion with Proteinase K.

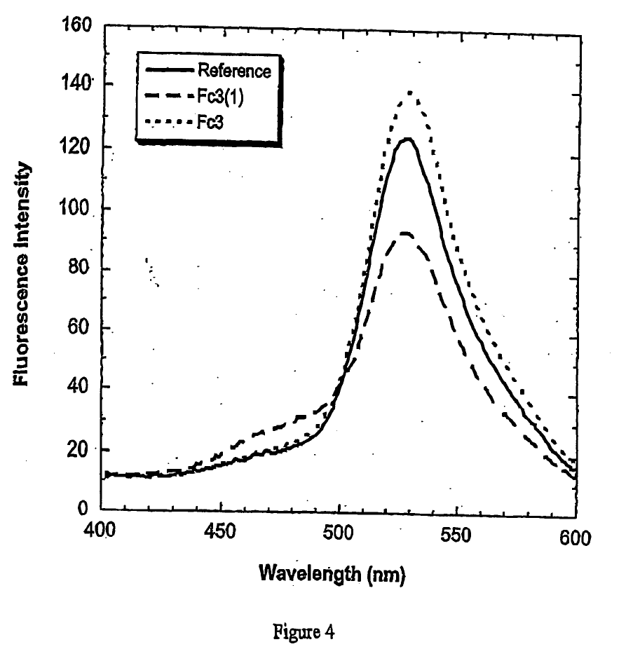

FIG. 4. Emission spectra of EDANS/NBDX-labelled B (N²³C)-ABD in PBS, pH 7.4, in the absence of target protein, in the presence of 300 nM Fc₃₍₁₎ and in the presence of 300 nM FC₃.

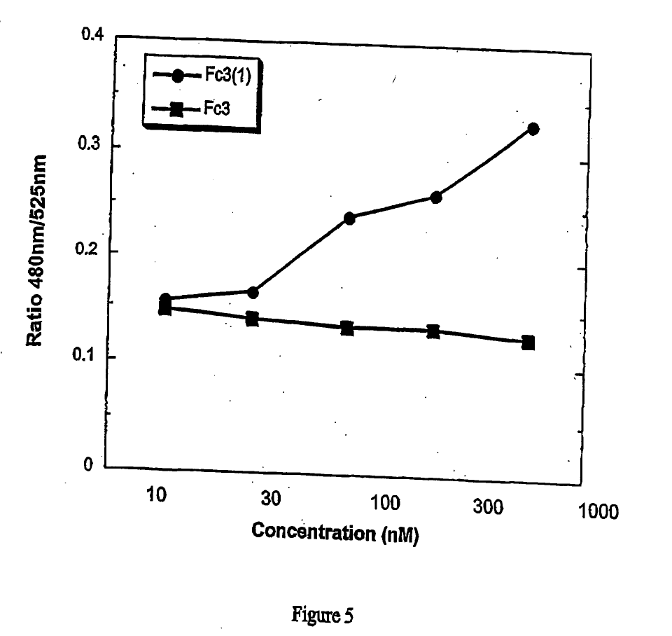

FIG. 5. Emission ratio 480 nm/525 nm for titration of EDANS/NBDX-labelled B(N²³C)-ABD with increasing concentrations of Fc₃₍₁₎ and Fc₃.

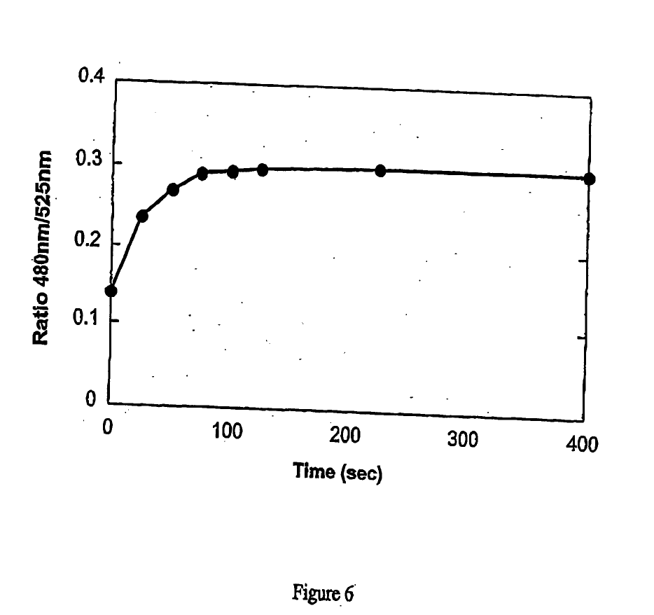

FIG. 6. Time-course for the shift in emission ratio 480 nm/525 nm of EDANS/NBDX-labelled B (N₂₃C)-ABD after the addition of Fc₃₍₁₎ to a final concentration of 500 nM.

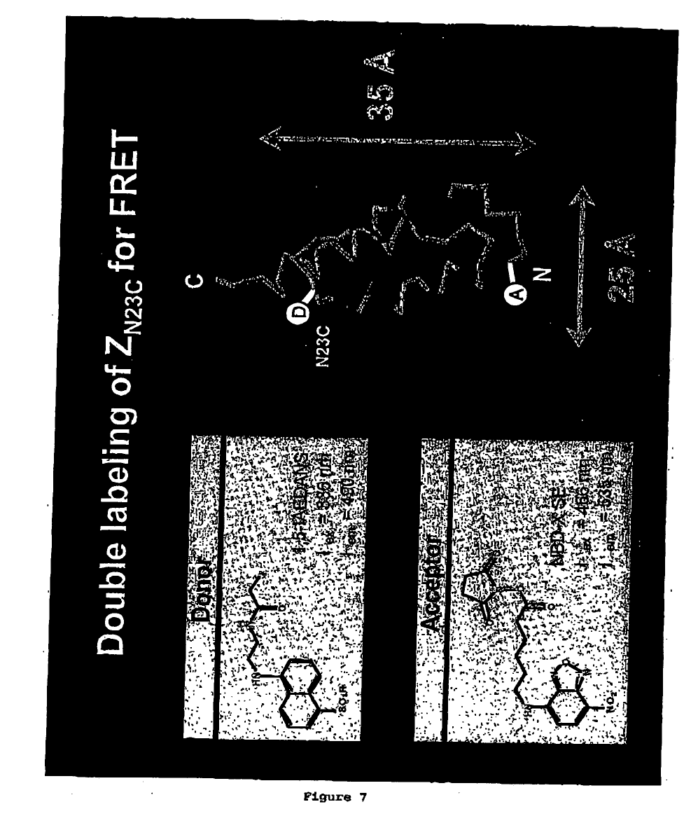

FIG. 7. A representation of the double labelling of the B/Z (N23C) derivative showing the donor and acceptor groups.

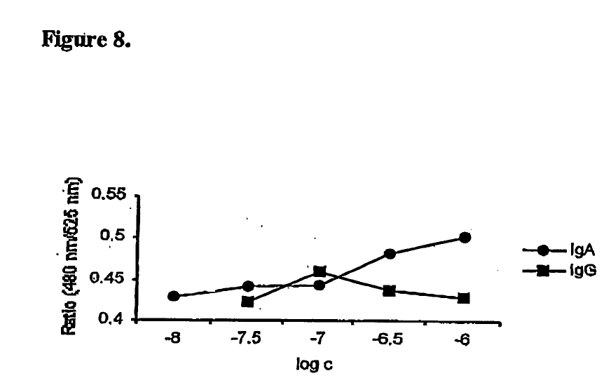

FIG. 8. A graph showing the Emission ratio 480 nm/525 nm for titration of EDANS/NBDX-labeled ZIgA (N23C)-ABD with increasing concentrations of human IgA or human polyclonal IgG.

### EXAMPLES

### Example 1

Experimental Procedures

Strains and Plasmids.

Escherichia coli strain RR1ΔM15 (Rüther et al, 1982) was used for cloning and mutagenesis and Escherichia coli strain RV308 (Maurer et al, 1980) was used for expression of soluble protein. The phagemid vector pKN1 (Nord et al, 1995) encoding the Omp A leader peptide, residues 44-58 of the Z domain and albumin-binding domain (ABD) was used for the cloning.

Site-Directed Mutagenesis.

An Asn²³Cys point mutation was introduced in the loop between helices 1 and 2 of domain B from staphylococcal protein A by overlap-extension PCR (Higuchi et al, 1988, Ho et al, 1989). PCR was carried out using AmpliTaq DNA Polymerase (Perkin-Elmer/Roche Molecular Systems, Inc., Branchburg, N.J.), the overlapping primers 5′- GTTTCGTTGTTCTTCGCATAAGTTAGGTAAATGTAAGATC- 3′ and 5′-GAAGAACAACGAAACGGCTTCATCCAAAGTTTA-3′ and the ZLIB3/ZLIB5 primer set (Nord et al, 1995). Fusion PCP products were gel purified and digested by Nhe I (MBI Fermentas, Vilnius, Lithuania) and Mlu I (New England Biolabs, Beverly, Mass.), followed by ligation to Nhe I/BsmB I (New England Biolabs) -digested pKN1 vector using T4 DNA ligase (MBI Fermentas). The ligated plasmid was transformed into E. coli RR1ΔM15 and the mutation was verified by DNA sequencing using NOKA2 and RIT27 as sequencing primers (Nord et al, 1995) and the MegaBACE 1000 DNA Sequencing System (Molecular Dynamics/Amersham Pharmacia Biotech, Sunnyvale, Calif.).

Protein Expression and Purification.

The plasmid was introduced into the host strain E. coli RV308 for production of protein. A single colony was inoculated in 10 ml Tryptic Soy Broth (TSB) (Merck KGgA, Darmstadt, Germany) supplemented with 100 μg ml⁻¹ ampicillin and shaken at 37° C. over night. The culture was back diluted 1:100 in a 500 ml culture of TSB supplemented with 100 μg ml⁻¹ ampicillin and 5 g 1⁻¹ yeast extract (Fould Springer, Maisons-Alfort, France) and grown in a 37° C. shaker until OD₆₀₀=1 was reached. Protein expression was induced by the addition of 1 mM IPTG and the culture was shaken at room temperature for 22 h. The cells were harvested by centrifugation at 4,000×g, +4° C., for 15 min and the pellet resuspended in TST buffer (25 mM Tris-HCl, pH 8.0, 1 mM EDTA, 200 mM NaCl, 0.05% (w/v) Tween 20). The periplasmic proteins were released by 3 freeze-thaw cycles and a clear fraction was obtained by centrifugation at 17,000×g, +4° C. for 20 min followed by filtration of the supernatant through a 0.45 μm filter. The ABD fusion protein was purified by HSA-sepharose affinity chromatography as described (Nygren et al, 1988) and the purity of the protein checked by 20% SDS-PAGE using the Phast system (Amersham Pharmacia Biotech, Uppsala, Sweden) and Coomassie Brilliant Blue staining.

Protein Modification.

Prior to labeling, the protein was dissolved in PBS, pH 7.4. The protein concentration was adjusted to 1 mg ml⁻¹ after estimation by measuring the OD₂₈₀ using the extinction coefficient 0.379 ml mg⁻¹ cm⁻¹. To reduce intermolecular disulfide bonds, the protein was incubated with 20 mM DTT (Sigma-Aldrich Chemie Gmbh, Steinheim, Germany) for 3 h at room temperature. DTT was removed by dialysis of the protein against degassed PBS, pH 7.4, using Spectra/Por^{R} dialysis tubes (Spectrum Medical Industries, Inc., Los Angeles, Calif.) with a cutoff of 3,500 Da. The reduced protein was labeled with N-iodoacetyl-N′-(5-sulfo-1-naphtyl) ethyl-enediamine (1,5-IAEDANS) or iodoacetamide, both purchased from Sigma-Aldrich, A 100-fold molar excess of thiol-reactive probe from a stock solution of 25 mg ml⁻¹ in DMSO (Sigma-Aldrich) was added to the dialysed protein and stirred at room temperature for 3 h, protected from light. The reaction was stopped by the addition of β-mercaptoethanol (Merck) at a 20-fold molar excess relative to the probe. The labeled protein was subsequently separated from excess probe and β-mercaptoethanol by gel filtration using a PD10 Sephadex G-25 column (Amersham Pharmacia Biotech) equilibrated with 5 mM NH₄OAc, pH 5.5, followed by lyophilization. For the introduction of a second label the lyophilized protein was dissolved in PBS, pH 7.0, at a concentration of 1 mg ml⁻¹, based on the original protein determination. A 10-fold molar excess of succinimidyl 6-(N-(7-nitrobenz-2-oxa-1,3-diazol-4-yl)amino)hexanoate (NBD-X, SE), purchased from Molecular Probes (Eugene, Oreg., USA), from a stock solution of 2 mg ml⁻¹ in DMSO was added and the solution stirred at room temperature for 15 min, protected from light. 150 mM hydroxylamine was added to the solution and stirring continued for an additional 30 min. The labeled protein was purified by gel filtration as described above, followed by lyophilization.

Purification and Analysis of Labeled Protein.

The labeled protein was purified by preparative RP-HPLC using a 4.6×150 mm column with polystyrene/divinyl benzene matrix and 5 μm particle size (Amersham Pharmacia Biotech). A flow rate of 1 ml min⁻¹ and an elution gradient of 35-40% B in 20 min, where solvent A: 0.1% TFA-H₂O and solvent B: 0.1% TFA-CH₃CN, was used for purification of labeled B(N²³C)-ABD. The same conditions were used for purification of the protein labeled with NBD-X and with the thiol blocked with iodoacetamide. The purified fractions were lyophilized and dissolved in PBS, pH 7.4. All purified fractions were analyzed by using 20% SDS-PAGE Phast analysis, in order to verify the purity and estimate the concentration of the protein. The fractions were analyzed by fluorescence spectroscopy (see below) to determine the presence of the fluorophores. Purified, doubly labeled B (N²³C)-ABD was further analyzed by mass spectrometry and amino acid analysis. The molecular weight was determined by MALDI-MS to estimate the degree of protein labeling. Amino acid analysis was used to determine the protein concentration, which was subsequently used for calculation of the binding affinity.

Target Proteins.

Fc₃₍₁₎ and Fc₃ were produced and purified as described (Jendeberg et al, 1997). Lyophilized protein was refolded by dissolving the protein in 6 M guanidinine hydrochloride-PBS followed by dialysis over night at 4° C., against PBS, pH 7.4.

Binding Analysis.

Retained binding of the labeled protein to its corresponding target protein was verified by biosensor analysis using a BIAcor™ 2000 instrument (Biacore AB, Uppsala, Sweden). Fc₃₍₁₎ and Fc₃ were immobilized by succinimide-mediated coupling to a carboxylated dextran layer of a CM5 sensor chip, according to the supplier's recommendations. In order to determine the combined effects of the mutation and fluorescent labelling on the binding affinity, the dissociation constant of the labeled B (N²³C)-ABD mutant was measured using wild-type Z-ABD as a reference. The binding analyses were carried out at 25° C., with a flow rate of 5 μl min⁻¹ and a sample volume of 20 μl. HBS (10 mM HEPES, pH 7.4, 0.15 M NaCl, 3.4 mM EDTA and 0.005% Surfuctant P20 (Biacore AB)) was used as the running buffer. The chip was regenerated by injection of 50 mM HCl. Binding curves were obtained by injection of protein of concentrations from 50 nM to 1.8 μM and the dissociation constant of the binding interaction was determined using the BIAevaluation software (Biacore AB).

Fluorescence Spectroscopy.

Fluorescence spectra were recorded using a Perkin-Elmer LS 50B fluorimeter (Perkin-Elmer Instruments, Norwalk, Conn.). The excitation wavelength was 336 nm and the fluorescence emission was scanned from 400 nm to 600 nm. A slit width of 10 nm was used both for the excitation and emission. Measurements were carried out in a semi-micro fluorescence cell with a light path of 10×4 mm (Hellma GmbH & Co., Müllheim/Baden, Germany). All spectra were recorded in PBS, pH 7.4. In the binding assays the concentration of the labeled binding protein was kept constant at 270 nM, with the concentration of target protein titrated from 10 nM to 1 μM. Proteolysis experiments were carried out by recording the fluorescence emission spectra of a protein solution in PBS, pH 7.4, before and after proteolysis. Proteolytic digestion was performed by adding 25 μg Proteinase K to the cuvette, followed by incubation at 37° C. for 30 min. Time course experiments were carried out by adding target protein to a final concentration of 500 nM to a 270 nM solution of labeled protein and measuring the fluorescence emission at 480 nm and 525 nm over a 10 min period of time.

Results

Preparation and Characterization off Labeled Protein.

To facilitate site-specific labeling of the protein, an asparagine residue at position 23 of the B domain was substituted for a unique cysteine residue (see FIG. 2). The mutant protein was found to express in E. coli at levels similar to the wild-type protein. SDS-PAGE analysis of purified protein showed the presence of dimers, consistent with the formation of intermolecular disulfide bonds in the periplasm. Coupling of the donor fluorophore (1.5-IAEDANS) to the introduced thiol was carried out wish a high excess of probe and the extent of labeling was shown by analytical RP-HPLC to vary between 50-100%. Coupling of the acceptor fluorophore (NBD)-X, SE) was carried out with a 10-fold excess of probe at low pH, in order to selectively label the N-terminal α-amino group, which has a lower pKₐ than the α-amino group of the lysine residues in the protein. The extent of labeling was monitored by analytical RP-HPLC and the reaction stopped by the addition of hydroxylamine, to avoid extensive labeling of the lysine residues. Labeled protein was purified by preparative RP-HPLC. Fluorescence spectra confirmed the presence of both fluorophores in the purified fraction and mass spectrometry verified that the protein was labeled with only one donor and one acceptor molecule. The calculated molecular weight of the B (N²³C)-ABD protein labeled with one donor molecule and one acceptor molecule is 14,086 Da and the molecular weight determined experimentally by MALDI-MS was 14,074 Da, which is within the error of the instrument (+/−0.1%). Biosensor binding analysis showed that the affinity for FC₃₍₁₎ was only marginally lower for labeled B (N²³C)-ABD protein (K_{d}=200 nm) compared to that of unlabeled, wild-type Z-ABD (K_{d}=90 nM).

Fluorescence Spectroscopy of Labeled B (N²³C)-ABD).

The fluorescence emission spectrum of EDANS/NBD-X-labeled B (N²³C)-ABD in the absence of target protein is shown in FIG. 3a (solid line). Excitation at 336 nm gives rise to a major peak of acceptor (NBD-X) emission with a maximum at 525 nm with a small shoulder of donor (EDANS) emission with a maximum at 490 nm. The presence of the NBD-X emission peak in the spectrum can be explained either by direct excitation of the fluorophore at 336 nm or by fluorescence resonance energy transfer from the donor to the acceptor. In order to investigate the contributions from different mechanisms, the labeled protein was non-specifically digested by Proteinase K and the emission spectra recorded before and after proteolysis (Epe et al, 1983). As reference, mono-labeled protein was prepared and treated in the same manner. In FIG. 3a, the spectra of EDANS/NBD-X-labeled protein before and after proteolysis is shown. After spatial separation of the two fluorophores by digestion of the protein, intramolecular fluorescence resonance energy transfer is abolished, and the result is a marked increase in donor fluorescence. In contrast, the spectra of the protein labeled only with donor fluorophore show that after proteolysis of the protein, the donor fluorescence is slightly lower (~20% decrease) (see FIG. 3b). Since the donor molecule is more accessible after proteolysis as compared to when bound to the intact protein, the weaker fluorescence could probably be explained by increased quenching by water molecules. The spectra of the protein labeled only with acceptor fluorophore show that the quantum yield of the free acceptor is dramatically lower than when bound to the protein (~80% decrease) (see FIG. 3c). It is known that the NBD fluorophore is sensitive to the environment and generally has a higher quantum yield when bound to a protein or in an apolar solvent than when free in aqueous solution (Kenner & Aboderin, 1971). Taken together, the proteolysis experiments with the mono and doubly labeled proteins indicate that fluorescence resonance energy transfer occurs in the protein labeled with both donor and acceptor. The acceptor emission in the doubly labeled protein could partly be due to direct excitation of the acceptor fluorophore, but the weak donor emission strongly suggests that energy is also transferred from the excited donor to the acceptor.

Fluorescence spectra of EDANS/NBD-X-labeled B(N²³C)-ABD in the presence and absence of target protein were recorded in order to determine how binding of target protein would affect the fluorescence signal (see FIG. 4). In an earlier study, protein A was shown to bind to the Fc region of human IgG subclass 3, but not IgG subclass 1 (Jendeberg et al, 1997). Engineering of recombinant Fc fragments showed that two amino acid substitutions in Fc₃ were sufficient to restore the protein A-binding capacity of Fc₁. In this study the engineered version Fc₃₍₁₎ was used as target protein and Fc₃ was used as a negative control for non-specific protein effects. It was shown that in the presence of Fc₃₍₁₎, which specifically binds to the labelled protein, the donor emission at 480 nm increases, whereas the acceptor emission at 525 nm decreases. This can be explained by a decrease in FRET between the donor and acceptor groups in the presence of target protein, which could be mediated by a conformational change in the labelled protein upon binding to its target. The spectrum of the labelled protein in the presence of the control, non-binding, protein Fc₃ shows a small increase in fluorescence emission at both 480 nm and 525 nm. When the ratio of fluorescence emission at 480 nm and 525 nm is calculated, the same value is obtained in the absence of target protein as in the presence of Fc₃, whereas the ratio is increased in the presence of Fc₃₍₁₎. Since Fc₃₍₁₎ and Fc₃ only differ in two positions, the different effects on the fluorescence emission is due to the specific binding of Fc₃₍₁₎ to the labelled protein, and not to other factors, such as different effects on the polarity of the medium, which could be expected if two proteins with different physical-chemical properties had been used.

Titration of EDANS/NBD-X-labelled B(N²³C)-ABD with increasing concentrations of Fc₃₍₁₎ and Fc₃ is shown in FIG. 5. The fluorescence emission 480 nm/525 nm ratio increases with increasing concentration of Fc₃₍₁₎, while the ratio stays constant with increasing concentration of Fc₃. These results indicate that the method can be used to quantify the presence of specific binders in an unknown sample. Saturation of the signal is not observed in the concentration range from 10 nM to 1 μM.

A time-course experiment showed that development of the signal is very fast and that the maximum is reached within less than 3 min after mixing the labelled binding protein with the target protein (see FIG. 6). The rapid development of the signal enables fast assessment of the presence of target protein in a sample using this binding assay.

### Example 2

Strains and Plasmids

pKN1-ZIgA (Gunneriusson et al., 1999), encoding an Z-affibody selected for binding to human IgA was used as template for PCR amplifications.

Site-Directed Mutagenesis.

For the mutagenesis of the ZIgA affibody, the same strategy as outlined above was used.

Protein Expression and Purification.

For the expression and purification of the ZIgA(N23C) affibody, the same strategy as outlined above was used.

Protein Modification.

For the modification of the ZIgA(N23C) affibody, the same strategy as outlined above was used.

Purification and Analysis of Labeled Protein.

For the purification and analysis of the ZIgA(N23C) affibody, the same strategy as outlined above was used.

Target Proteins.

Human polyclonal IgG was obtained from Pharmacia, Stockholm, Sweden and human IgA was obtained by affinity chromatography purification from normal human plasma (Karolinska Hospital, Sweden)

Fluorescence Spectroscopy.

Fluorescence spectroscopy analyses for the ZIgA(N23C) affibody, was performed according to the strategy as outlined above.

A second affinity protein developed using combinatorial protein engineering and showing selective binding to human IgA (Gunneriusson et al., 1999; Nord et al., 1997) was investigated according to the same principles as described for the B (N23C)-ABD protein in Example 1. Here, the correspondingly mutated and doubly labeled binding protein ZIgA(N23C), also expressed as an ABD-fusion protein was tested in fluorescence spectroscopy detection of human IgA, using human polyclonal IgG as control . The results (FIG. 8) showed that the emission ratio 480 nm/525 nm for the titration with the IgG control did not change significantly in contrast, the emission ratio 480 nm/525 nm for the titration with human IgA, corresponding to the target for the modified affibody investigated, showed to increase when higher concentrations of the target were used. This shows that the described detection principle is applicable also to proteins developed by combinatorial protein engineering, performed in order to engineer their binding specificities.

References

Förster, T. (1948) Zwischenmolekulare Energiewanderung und Fluoreszenz, Ann. Physik. 2, 55-75.

Miyawaki, A., Llopis, J., Heim, R., McCaffery, J. M., Adams, J. A., Ikura, M & Tsien, R. Y. (1997) Fluorescent indicators for Ca²⁺ based on green fluorescent proteins and calmodulin. Nature 388, 882-887.

Cotton, G. J. & Muir, T. W. (2000) Generation of a dual-labelled fluorescence biosensor for Crk-II phosphorylation using solid-phase expressed protein ligation. Chemistry & Biology 7, 253-261.

Nomanbhoy, T. & Cerione, R. A. (1999) Fluorescence assays of Cdc42 interactions with target/effector proteins. Biochemistry 38, 15878-15884.

Ueda, H., Kubota, K., Wang, Y., Tsumoto, K., Mahoney, W., Kumagai, I. & Negamune, T. (1999) Homogeneous noncompetitive immunoassay based on the energy transfer between fluorolabeled antibody variable domains (open sandwich immunoassay). BioTechniques 27, 738-742.

Arai, R., Ueda, H., Tsumoto, K., Mahoney, W. C., Kumagai, I. & Nagamune, T. (2000) Fluorolabeling of antibody variable domains with green fluorescent protein variants: application to an energy transfer-based homogeneous immunoassay. Prot. Eng. 13, 369-376.

Nord, K., Nilsson, J., Nilsson, B., Uhlén, M. & Nygren, P. -Å. (1995) A combinatorial library of an α-helical bacterial receptor domain. Prot. Eng. 8, 601-608.

Nord, K., Gunneriusson, E., Ringdahl, J., St\dot{a}hl, S., Uhlén, M. & Nygren, P.-Å. (1997) Binding proteins selected from combinatorial libraries of an α-helical bacterial receptor domain. Nature Biotechnol. 15, 772-777.

Nord, K., Gunneriusson, E., Uhlén, M. & Nygren, P.-Å. (2000) Ligands selected from combinatorial libraries of protein A for use in affinity capture of apolipoprotein A-1_{M} and Taq DNA polymerase. J. Biotechnol. 80, 45-54.

Gunneriusson, E., Nord, K., Uhlén, M. & Nygren, P.-Å. (1999) Affinity maturation of a Tag DNA polymerase specific affibody by helix shuffling . Prot, Eng. 12, 873-878.

Hansson, M., Ringdahl, J., Robert, A., Power, U., Goetsch, L., Nguyen, T. N., Uhlén, M., St\dot{a}hl, S. & Nygren, P.-Å. (1999) An in vitro selected binding protein (affibody) shows conformation-dependent recognition of the respiratory syncytial virus (RSV) G protein. Immunotechnology 4, 237-252.

Rüther, U. (1982) pUR250 allows rapid chemical sequencing of both DNA strands of inserts. Nucleic Acids Res. 10, 5765-5772.

Maurer, R., Meyer, B. & Ptashne, M. (1980) Gene regulation at the right operator (OR) bacteriophage lambda. I. OR3 and autogenous negative control by repressor. J. Mol. Biol. 139, 147-161.

Higuchi, R., Krummel, B, & Saiki, R. K. (1988) A general method for in vitro preparation and specific mutagenesis of DNA fragments: study of protein and DNA interactions. Nucleic Acids Res. 16, 7351-7367.

Ho, S. N., Hunt, H. D., Horton, R. M., Pullen, J. K. & Pease, L. R. (1989) Site-directed mutagenesis by overlap extension using the polymerase chain reaction. Gene 77, 51-59.

Nygren, P.-Å., Eliasson, M., Abrahmsén, L., Ulhén, M. & Palmcrantz, E. (1988) Analysis and use of the serum albumin binding domains of streptococcal protein G. J. Mol. Recognit. 1, 69-74.

Jendeberg, L., Nilsson, P., Larsson, A., Denker, P., Uhlén, M., Nilsson, B. & Nygren, P.-Å. (1997) Engineering of Fc1 and Fc3 from human immunoglobulin G to analyse subclass specificity for staphylococcal protein A. J. Immunol. Methods 201, 25-34.

Gouda, H., Torigoe, H., Saito, A., Sato, M., Arata, Y & Shimada, I. (1992) Three-dimensional solution structure of the B domain of staphylococcal protein A: comparisons of the solution and crystal structures. Biochemistry 31, 9665-9672.

Epe, B., Steinhäiser, K. G. & Woolley, P. (1983) Theory of measurement of Förster-type energy transfer in macromolecules. Proc. Natl. Acad. Sci. USA 80, 2579-2583.

Kenner, R. A. & Aboderin, A. A. (1971) A new fluorescent probe for protein and nucleoprotein conformation. Binding of 7-(p-methoxybenzylamino)-4-nitrobenzoxadizole to bovine trypsinogen and bacterial ribosomes. Biochemistry 10, 4433-4440.

Silzel, J. W., Cercek, B., Dodson, C., Tsay, T. & Obremski, R. J. (1998) Mass-sensing, multianalyte microarray immunoassay with imaging detection. Clin. Chem. 44, 2036-2043.

Nilsson, B., Moks, T., Jansson, B., Abrahmsén, L., Elmblad, A., Holmgren, E., Henrichson, C., Jones, T. A. & Uhlén, M. (1987) A synthetic IgG-binding domain based on staphylococcal protein A. Prot. Eng. 1, 107-112.

Kronball, G., and Jonsson, K. (1999) Receptins: a novel term for an expanding spectrum of natural and engineered microbial proteins with binding properties of mammalian proteins. J. Mol. Recognit. 12(1), 38-44.

Gunneriusson, E., Samuelson, P., Ringdahl, J., Grönlund, H., Nygren, P.-Å. and St\dot{a}hl, S. (1999) “Staphylococcal surface display of Immunoglobulin A (IgA) and IgE-specific in vitro-selected binding proteins based on Staphylococcus aureus protein A ” Appl. Envir. Microbiol. 65, 4134-4140.

1. A method of detecting the presence of a target molecule in a sample which comprises contacting said sample with a detector protein, said detector protein:
(a) being a binding partner for said target molecule;
(b) being derivatized by two fluorescent reporter groups; and
(c) comprising a combinatorial protein,
the energy transfer between said reporter groups undergoing a detectable change on binding of the target molecule to said detector protein.

2. A method as claimed in claim 1 wherein changes in the fluorescence emission ratio from the two reporter groups depend on the amount of target molecule present in the sample, allowing quantitative information regarding the target in the sample to be obtained.

3. A method as claimed in claim 1 wherein the fluorescent reporter groups are non-proteinaceous groups.

4. A method as claimed in claim 1 wherein the detector protein comprises a single binding domain which is derivatized by two fluorescent reporter groups and which is a binding partner for said target molecule.

5. A method as claimed in any preceding claim wherein the detector protein comprises a combinatorial protein derived from staphylococcal protein A.

6. A method as claimed in claim 5 wherein the detector protein comprises a combinatorial protein derived from the B domain of staphylococcal protein A.

7. A method as claimed in any preceding claim wherein the detector protein haw been modified by fluorescent reporter groups in a site-specific manner.

8. A method as claimed in any preceding claim wherein no more than one of the fluorescent reporter groups is located at the N or C terminus of the detector protein.

9. A method as claimed in claim 8 wherein one of the fluorescent reporter groups is located at the N or C terminus of the detector protein.

10. A method as claimed in any preceding claim wherein one of the reporter groups is capable of being covalently attached to only one of the internal amino acid residues in the binding domain.

11. A method as claimed in claim 10 wherein said amino acid residue is a cysteine residue.

12. A method as claimed in any preceding claim wherein the detector protein undergoes a conformational change as binding to the target molecule.

13. A method as claimed in any preceding claim wherein the target molecule is a protein, protein derivative or fragment, polypeptide or peptide.

14. A method of preparing a detector protein or use in the detection of a target molecule in a sample, said method comprising:
(a) selection of a combinatorial binding protein capable of binding to said target molecule,
(b) optionally introducing into the protein identified in step (a) one or more amino acid residues which can be derivatized by a fluorescent reporter group, and
(c) labelling the binding protein either simultaneously or sequentially with two fluorescent reporter groups.

15. A detector protein molecule having a binding sites for a target molecule and having covalently attached thereto two fluorescent reporter groups, the energy transfer between said reporter groups undergoing a detectable change on binding of the target molecule to said detector protein, said detector protein comprising a combinatorial protein.


SEQUENCE LISTING (2 sequences)

SEQ ID NO: 1 - DNA, 40 bp, Artificial Sequence
  Primer of Staphylococcal protein A.
gtttcgttgttcttcgcataagttaggtaaatgtaagatc

SEQ ID NO: 2 - DNA, 33 bp, Artificial Sequence
  Primer of Staphylococcal protein A.
gaagaacaacgaaacggcttcatccaaagttta